(19) United States
(12) Patent Application Publication
(10) Pub. No.: US 2023/0180855 A1
(43) Pub. Date: Jun. 15, 2023

(54) FLEXIBLE VAPORIZER

(71) Applicant: Benyamin Abehasera, Hallandale Beach, FL (US)

(72) Inventor: Benyamin Abehasera, Hallandale Beach, FL (US)

(21) Appl. No.: 18/150,326

(22) Filed: Jan. 5, 2023

Related U.S. Application Data
(63) Continuation-in-part of application No. 17/549,360, filed on Dec. 13, 2021, now abandoned.

(51) Int. Cl. A24F 40/85; A24F 7/00; A24F 40/50; A24F 40/40; A61L 9/20

(52) U.S. Cl. CPC A24F 40/85; A24F 7/00; A24F 40/40; A24F 40/50; A61L 9/20; A61L 2209/12

(57) ABSTRACT

A flexible electronic smoking device, such as a vaporizer, has a flexible main body with one end having a mouthpiece and the other end having a recess. The mouthpiece is designed to be received in and retained by the recess such that the recess functions as a cap. The recess includes sanitizing LEDs that can be turned on by a manual switch or automatically when the mouthpiece is at least partially received in the recess. The main body includes a flexible, single-cell, ribbon-type battery. Also, the main body can include a pod containing an inhalant material container, a heating element, a microcontroller, and a switch.

### CROSS REFERENCE TO RELATED APPLICATIONS

This application is a continuation in part of U.S. App. Ser. No. 17/549,360 filed on Dec. 13, 2021.

### BRIEF DESCRIPTION OF THE DRAWINGS

FIG. 1 is a perspective view of the flexible vaporizer in an extended position.

FIG. 2A is a perspective view of the flexible vaporizer in a partially closed position.

FIG. 2B is a perspective view of the flexible vaporizer in a closed position.

FIG. 3 is a schematic of the microcontroller and various electronic components of the flexible vaporizer.

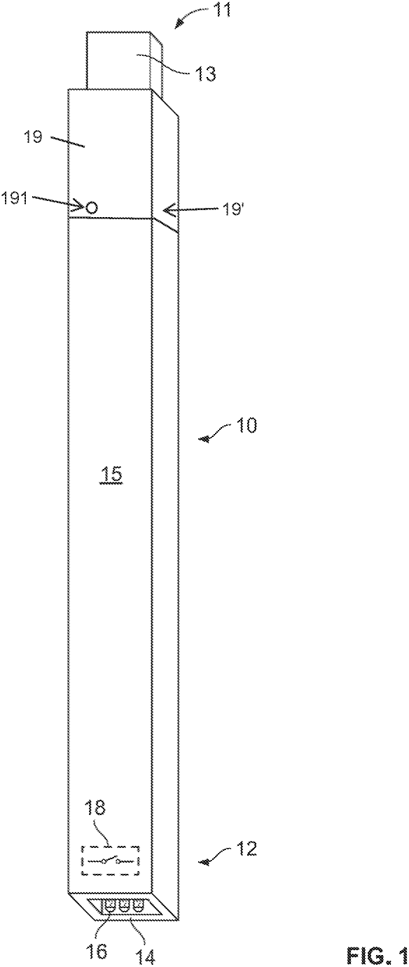

It will be recognized that some or all of the figures are schematic representations for purposes of illustration and do not necessarily depict the actual relative sizes or locations of the elements shown. The figures are provided for the purpose of illustrating one or more embodiments of the invention with the explicit understanding that they will not be used to limit the scope or the meaning of the claims.

### DETAILED DESCRIPTION OF THE INVENTION

In the following description, for the purposes of explanation, numerous specific details are set forth to provide a thorough understanding of the invention. It will be apparent, however, to one skilled in the art that the invention may be practiced without some of these specific details. Throughout this description, the embodiments and examples shown should be considered as exemplars, rather than as limitations on the invention. That is, the following description provides examples, and the accompanying drawings show various examples for the purposes of illustration. However, these examples should not be construed in a limiting sense as they are merely intended to provide examples of the invention rather than to provide an exhaustive list of all possible implementations of thereof.

Specific embodiments of the invention will now be further described by the following, non-limiting examples which will serve to illustrate various features. The examples are intended merely to facilitate an understanding of ways in which the invention may be practiced and to further enable those of skill in the art to practice the invention. Accordingly, the examples should not be construed as limiting the scope of the invention. In addition, reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner in one or more embodiments.

Referring to the Figures, shown is an electronic smoking device 10 configured as a vaporizer having a proximal end 11 and an opposing distal end 12. The proximal end 11 delimits a mouthpiece 13 whereon a user places his/her mouth to take a drag from the electronic smoking device. The distal end 12 comprises a recess 14 configured as a cap for the mouthpiece 13 as further described herein. In some embodiments, disposed inside, adjacent to, or about the recess 14 are one or more light-emitting diodes (LEDs) 16 which are configured to emit light of a wavelength in the range of 100-280 nm and, more particularly, in the range of 200-280 nm, which is considered the UVC range effective for neutralizing microorganisms such as bacteria, viruses, and the like.

The main body 15 of the vaporizer is generally malleable and flexible enabled through the use a flexible outer housing comprised of flexible plastic, rubber, the combinations thereof. In some embodiments, inside the main body 15 is a single-cell ribbon-type, flexible, rechargeable battery such as, for example purposes only, a 3.7 v 130 mah “Ultrathin Ultra Safe Flexible Lithium Ceramic Battery.” This configuration of battery, i.e. single-cell ribbon-type, avoids the need for multiple batteries or stacked batteries and optimizes articulation and flexibility of the main body 15. Additionally, the use of a single-cell battery enables the main body 15 to have a unibody construction such that, in some embodiments, the main body 15 is manufactured (for example, by extrusion) as a single piece.

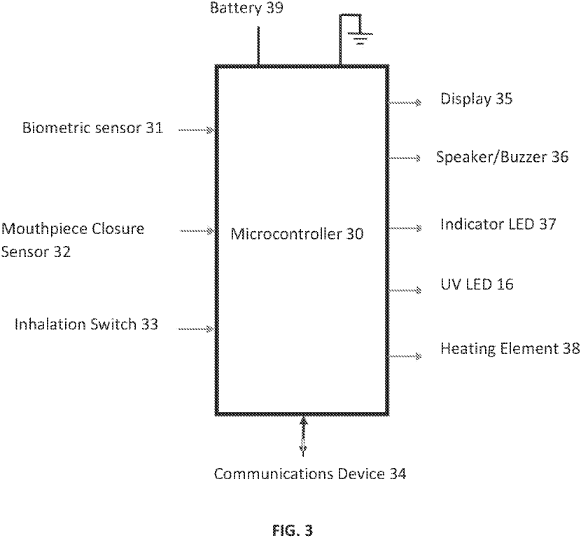

The main body 15 also includes other components of an electronic smoking device including an inhalant material container, a heating element, a microcontroller, and a switch which may be configured as a diaphragm to activate the heating element when the user takes a drag (e.g. draws air through the device) or the switch may be connected to an external button to activate the heating element manually. With reference to FIG. 3, the microcontroller 30 can be in electrical communication with and control various components of the device 10, including a biometric sensor 31 (e.g., a fingerprint lock), a mouthpiece closure sensor 32, a switch 33 (as described above, for inhalation detection), a wired or wireless communications device 34, a display 35, a speaker or buzzer 36, an indicator such as an LED 37, the UV LEDs 16, and the heating element 38. These elements, either discretely or by way of the microcontroller, are powered by the battery 39, which battery is the single-cell, ribbon-type as described above.

In some embodiments, the foregoing components of the electronic smoking device are configured as a pod or cartridge 19 located about the proximal end 11 of the device 10 adjacent to and integrated with the mouthpiece 13. In some embodiments the pod 19 includes air intake apertures 191 at the distal end 19′ of the pod 19, which distal end is located substantially closer to the proximal end 11 of the device 10 than the distal end 12 of the device 10. The air intake apertures 191 are in fluid communication with the mouthpiece such that the pod 19 can pass fluid therethrough (e.g. air drawn through the device).

In some embodiments, the pod 19 comprises less than 50% of the entire length of the main body 15, biased toward the proximal end 11. In other embodiments the pod 19 comprises less than 25% of the entire length of the main body 15, biased toward the proximal end 11. In other embodiments the pod 19 comprises less than 10% of the entire length of the main body 15, biased toward the proximal end 11. In other embodiments the pod 19 comprises less than 5% of the entire length of the main body 15, biased toward the proximal end 11. These configurations provide increasing flexibility and articulation of the main body 15. In some embodiments, the pod 19 is removably connected to the main body 15 by a snap-fit, threaded connector, magnetic connector or other releasable connector. In some embodiments, the releasable connector is configured to also function as an electrical contact to transmit current from the battery in the main body 15 to the electronic smoking device components of the pod 19

In some embodiments, the internal, flexible, ribbon-type battery supplies power to the heating element, microcontroller, and the LEDs 16. In some embodiments, the microcontroller is programmed with logic to control various functions of the electronic smoking device 10. Further still, in some embodiments, the recess 14 includes a light switch or proximity sensor 18 that is configured to turn on the LEDs automatically when the recess 14 is placed (partially or otherwise) over the mouthpiece 13.

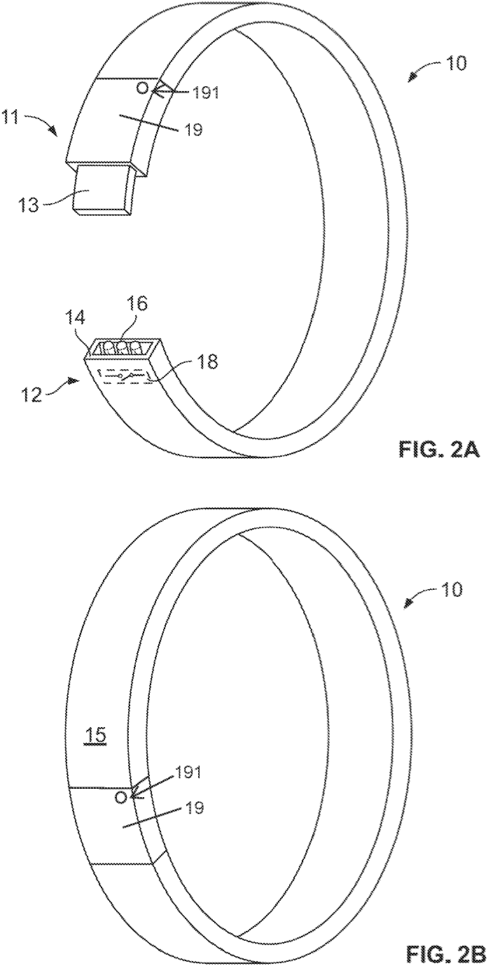

As shown in FIGS. 2A and 2B, the electronic smoking device 10 is flexible such that the distal 11 and proximal 12 ends can be brought together into a “closed” position whereby the mouthpiece 13 is received into and retained by the recess 14. In some embodiments, doing so results in the electronic smoking device 10 forming a ring or annulus although other configurations are possible depending on the dimensions of the components of the device. In some embodiments, the engagement of the recess 14 to the mouthpiece 13 comprises a snap fit or interference fit. Also, as noted, in some embodiments, the LEDs 16 activate automatically upon at least partially engagement of the recess 14 over the mouthpiece 13 such that the sanitizing action of the LEDs 16 is automatic when electronic smoking device 10 is in a closed (or partially closed) position. IN some embodiments, the automatic activation of the LEDs 16 is accomplished through the mouthpiece closure sensor 32 which is in communication with the LEDs 16 via the microcontroller 30.

Accordingly, it is appreciated and understood that the present invention provides an electronic smoking device 10 configured to have an integrated, sanitizing cap that greatly enhances the overall hygiene of the device and that prevents the introduction of unwanted materials, chemical, bacteria, and virus when the device is not being used.

It is to be noticed that the term “comprising,” used in the claims, should not be interpreted as being limitative to the means listed thereafter. Thus, the scope of the expression “a device comprising means A and B” should not be limited to devices consisting only of components A and B. It means that with respect to the present invention, the only relevant components of the device are A and B. Put differently, the terms “including”, “comprising” and variations thereof mean “including but not limited to”, unless expressly specified otherwise.

Similarly, it is to be noticed that the term “coupled”, also used in the claims, should not be interpreted as being limitative to direct connections only. Thus, the scope of the expression “a device A coupled to a device B” should not be limited to devices or systems wherein an output of device A is directly connected to an input of device B. It means that there exists a path between an output of A and an input of B which may be a path including other devices or means.

The enumerated listing of items does not imply that any or all of the items are mutually exclusive, unless expressly specified otherwise. The terms “a”, “an” and “the” mean “one or more”, unless expressly specified otherwise.

Elements of the invention that are in communication with each other need not be in continuous communication with each other, unless expressly specified otherwise. In addition, elements of the invention that are in communication with each other may communicate directly or indirectly through one or more other elements or other intermediaries.

One skilled in the art will appreciate that the present invention can be practiced by other than the above-described embodiments, which are presented in this description for purposes of illustration and not of limitation. The specification and drawings are not intended to limit the exclusionary scope of this patent document. It is noted that various equivalents for the particular embodiments discussed in this description may practice the invention as well. That is, while the present invention has been described in conjunction with specific embodiments, it is evident that any alternatives, modifications, permutations, and variations will become apparent to those of ordinary skill in the art in light of the foregoing description. Accordingly, it is intended that the present invention embrace all such alternatives, modifications and variations as fall within the scope of the appended claims. The fact that a product, process or method exhibits differences from one or more of the above-described exemplary embodiments does not mean that the product or process is outside the scope (literal scope and/or other legally-recognized scope) of the following claims.

What is claimed is:

1. An electronic smoking device, comprising
a flexible main body having a proximal end and a distal end, and containing a flexible, single-cell, ribbon-type battery;
the proximal end delimiting a mouthpiece and the distal end comprising a recess configured as a cap for the mouthpiece;
the recess including one or more light-emitting diodes (LEDs); and
wherein the main body is configured to be flexed such that the mouthpiece is received and retained in the recess.

2. The electronic smoking device of claim 1, wherein the one or more light-emitting diodes (LEDs) are configured to emit light of a wavelength effective for neutralizing bacteria and viruses.

3. The electronic smoking device of claim 2, wherein the wavelength comprises a range of 100-280 nm.

4. The electronic smoking device of claim 2, wherein the wavelength comprises a range of 200-280 nm.

5. The electronic smoking device of claim 1, wherein the one or more LEDs are in communication with a switch, the switch enabled to turn on the one or more LEDs.

6. The electronic smoking device of claim 5, wherein the switch is configured to activate the LEDs automatically upon at least partial insertion of the mouthpiece into the recess.

7. The electronic smoking device of claim 1, wherein the main body forms an annulus with the mouthpiece received at least partially in the recess.

8. The electronic smoking device of claim 1, wherein the main body includes a pod comprising an inhalant material container, a heating element, a microcontroller, and a switch.

9. The electronic smoking device of claim 8, wherein the heating element and the microcontroller are in electrical communication with the flexible, single-cell, ribbon-type battery.

10. The electronic smoking device of claim 8, wherein the pod is located about the proximal end thereof.

11. The electronic smoking device of claim 8, wherein the pod includes one or more air intake apertures in fluid communication with the mouthpiece.

12. The electronic smoking device of claim 8, wherein the microcontroller is in electrical communication with and controls a biometric sensor, a mouthpiece closure sensor, the switch, a communications device, a display, a speaker, an indicator, the one or more light-emitting diodes, and the heating element.

13. An electronic smoking device, comprising
a flexible main body having a proximal end and a distal end, and containing a flexible, single-cell, ribbon-type battery;
the proximal end delimiting a mouthpiece and the distal end comprising a recess, the mouthpiece configured to be received in and retained by the recess;
the recess including one or more light-emitting diodes (LEDs) configured to emit light of a wavelength effective for neutralizing bacteria and viruses; and
wherein the one or more LEDs are in communication with a switch, wherein the switch is enabled to activate the LEDs automatically upon at least partial insertion of the mouthpiece into the recess.

14. The electronic smoking device of claim 13, wherein the main body includes a pod comprising an inhalant material container, a heating element, a microcontroller, and the switch.

15. The electronic smoking device of claim 14, wherein the heating element and the microcontroller are in electrical communication with the flexible, single-cell, ribbon-type battery.

16. The electronic smoking device of claim 15, wherein the pod is located about the proximal end thereof.

17. The electronic smoking device of claim 16, wherein the pod includes one or more air intake apertures in fluid communication with the mouthpiece.

18. The electronic smoking device of claim 14, wherein the microcontroller is in electrical communication with and controls a biometric sensor, a mouthpiece closure sensor, the switch, a communications device, a display, a speaker, an indicator, the one or more light-emitting diodes, and the heating element.
